(19) United States
(12) Patent Application Publication
(10) Pub. No.: US 2026/0284036 A1
(43) Pub. Date: Sep. 24, 2026

(54) ORAL COMPOSITIONS CONTAINING METHOCINNAMOX

(71) Applicant: Board of Regents, The University of Texas System, Austin, TX (US)

(72) Inventor: Charles Patrick FRANCE, Austin, TX (US)

(21) Appl. No.: 19/472,304

(22) PCT Filed: Apr. 4, 2024
(86) PCT No.: PCT/US2024/022942

§ 371 Date: Oct. 3, 2025

Related U.S. Application Data
(60) Provisional application No. 63/494,031, filed on Apr. 4, 2023.

(51) Int. Cl. A61K 31/485; A61K 31/145; A61K 31/185; A61K 31/195; A61K 31/197; A61K 31/357; A61P 25/36

(52) U.S. Cl. CPC A61K 31/485; A61K 31/145; A61K 31/185; A61K 31/195; A61K 31/197; A61K 31/357; A61P 25/36

(57) ABSTRACT

Disclosed herein are orally administrable compositions including MCAM. The compositions are useful for treating and preventing opioid use disorder. The compositions and methods are also useful for treating alcohol use disorder, and other disorders associated with endogenous opioid production.

### CROSS-REFERENCE TO RELATED APPLICATION

This application claims the benefit of U.S. Provisional Application 63/494,031, filed Apr. 4, 2023, the contents of which are hereby incorporated in its entirety.

### STATEMENT OF GOVERNMENT SUPPORT

This invention was made with government support under R01DA048417 and UG3DA048387, awarded by the National Institute of Drug Abuse, National Institutes of Health. The government has certain rights in the invention.

### FIELD OF THE INVENTION

The invention relates to orally administrable compositions containing methocinnamox. The compositions are useful for treatment of opioid and alcohol use disorders.

### BACKGROUND

Opioids, including morphine, fentanyl, codeine, and oxycodone, have an essential medical role both as analgesics and anesthetics. Opioids reduce perception of pain by binding to opioid receptors in the central nervous system. These receptors include mu, kappa, sigma, delta, and epsilon subtypes. Mu and sigma receptors activate the dopaminergic mesolimbic system, triggering the release of dopamine and creating a feeling of euphoria and well-being. Nevertheless, opioids are also highly susceptible to abuse and are considered highly addictive. Opioid Use Disorder (OUD) is a neurological condition that arises as the consequence of repeated and compulsive use of opioid drugs. With prolonged use, opioids can cause several negative signs and symptoms besides addiction, including negative mood, muscle cramps, diarrhea, anxiety, and tremors. Additionally, as opioid use continues, the brain adapts to the drugs, causing increased tolerance and leading subjects to take higher doses of the drug. This, in turn, increases the risk of opioid overdose and death. Opioid use disorder, including overdose, can arise when opioids are used recreationally, i.e., non-medically prescribed opioid use disorder, and also when the opioids are used (at first) for a legitimate medical use which then leads to consumption of the drugs in non-prescribed doses (prescribed opioid use disorder).

Opioid overdose is a significant problem throughout the world; it affects recreational drug users and as well as individuals who abuse drugs, as well as patients treated with prescribed medications. Overdose deaths from fentanyl, heroin and other opioids have reached alarming numbers. In the United States, data from the National Center for Health Statistics showed a steady increase from 15,800 deaths in 2005, to 40,100 deaths in 2017. Drug overdose now accounts for over 100,000 deaths annually, most involving opioids, and the number of deaths is constantly increasing. Opioid intoxication is manifested by reduced consciousness and respiratory depression which may deteriorate to cardiac arrest and death. Overdose of opioids leads to depressed heart rate and breathing, leading to hypoxia. Hypoxia leads to short- and long-term effects on the central nervous system, including coma and permanent brain damage, which can result in the death of the hypoxic person. Overdoses of opioids, particularly heroin and fentanyl are very common. Fentanyl is a synthetic compound that is much more potent that morphine. Fentanyl analogs, which can be even more potent that fentanyl, further increase the risk of overdose if a user is unfamiliar with the strength of a particular analog in comparison with regular fentanyl or morphine. It is not unheard of for people to overdose the very first time they use powerful opioids. These risks are compounded when opioids are taken in combination with other substances, including alcohol or benzodiazepines.

In view of the potentially tragic outcome of overdose, a subject that has overdosed on an opioid requires urgent medical attention. The only medicines that can be employed to treat opioid overdoses effectively are opioid receptor antagonists, which act by binding to opioid receptors, displacing opioid agonists (like heroin) without eliciting opioid effects of their own, whether intended (e.g. euphoria) or unintended and/or potentially dangerous (including respiratory depression). Emergency administration of opioid antagonists can reduce (sometimes completely) the degree of opioid intoxication and, in essence, ‘reverse’ an opioid overdose.

The opioid antagonist, naloxone, can be used to treat overdose, and is available in the form of a single dose either as a liquid nasal spray (Narcan®; which is sprayed directly into one nostril), or as an auto-injector (Evzio®; which delivers drug by injection into the muscle or under the skin). When administered orally, naloxone undergoes significant first-pass metabolism and has an apparent bioavailability of 1-2%, and as such orally administrable compositions of naloxone are not effective to produce therapeutically effective systemic levels of the drug.

Naloxone is undoubtedly effective in helping to save lives. However, naloxone has a relatively short half-life. The short half-life can lead to re-narcotization, in which naloxone is cleared from the rescued victim while dangerous levels of opioids remain. In such an event, opioid intoxication symptoms can re-emerge, leading to cardiac arrest and death. As such, in about one third of overdose cases, two or more doses administered over time are needed to completely protect a subject from the harm of the overdose.

Beyond death and other medical catastrophes, chronic substance abuse places enormous burdens on society. Efforts to reduce the numbers of people that abuse alcohol and controlled substances such as opioids have been continuous and relatively unsuccessful. There is a relationship between alcohol intake and endogenous opioid production. Ethanol intake increases the synthesis and release of endogenous opioids (i.e., endorphins), which contributes to the positive reinforcement properties of ethanol. These findings have suggested the use of opioid receptor antagonists (such as naloxone or naltrexone) to prevent ethanol-induced analgesia, intoxication, and coma. Opioid antagonists, such as naltrexone, have been used to reduce the rate of relapse to heavy drinking in alcohol dependent patients.

Methocinnamox (“MCAM”) is long-acting and highly selective opioid receptor antagonist.

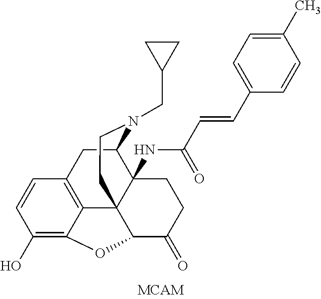 [SMILES: Cc1ccc(/C=C/C(=O)N[C@@]23CCC(=O)[C@@H]4Oc5c(O)ccc6c5[C@@]42CCN(CC2CC2)[C@@H]3C6)cc1]

Compared with other opioid receptor antagonists, MCAM binds to opioid receptors for extended periods of time, so that its effects are very long lasting compared with other antagonists. As such MCAM is an effective medication for treating opioid abuse, because of its long duration of action. A subject receiving MCAM is less likely to seek out opioids, because the MCAM effectively renders the subject immune to the euphoric effects of the opioid. MCAM is also useful for treating opioid overdose, because of its long duration of protection. Moreover, the risk of re-narcotization is much less likely when overdose is treated with MCAM than other antagonists.

There remains a need for improved orally active compositions for the treatment and prevention of opioid use disorder. There remains a need for orally active compositions for the treatment and prevention of substance abuse, including alcohol use disorder. There remains a need for orally active compositions for the treatment and prevention of opioid overdose.

### BRIEF DESCRIPTION OF THE FIGURES

FIG. 1A depicts the effects of orally administered methocinnamox (0.1, 0.32, and 1.0 mg/kg [HCl]) on the ventilatory depressant effects of fentanyl (subcutaneous) in four rhesus monkeys (TI, OL, ME, and LO). Under control conditions fentanyl decreased ventilation (minute volume [V_{E}]) in a dose-related matter in all four monkeys (filled circles, FIG. 1A). Orally administered (nasogastric intubation) methocinnamox antagonized the ventilatory depressant effects of fentanyl in all four monkeys, shifting the fentanyl dose-response curve rightward. Methocinnamox was effective the day after administration (triangles, FIG. 1A) and for up to 8 days after administration.

FIG. 1B depicts the effects of orally administered methocinnamox (100 mg/kg [base]) on the antinociceptive effects of fentanyl in groups of 2 male rats each (left panel, methocinnamox HCl; right panel, methocinnamox maleate). Under control conditions (filled circles), fentanyl increased the latency for rats to remove their tails from warm (50° C.) water, producing a maximum possible effect at a dose of 0.1 mg/kg. Orally administered (gastric intubation) methocinnamox antagonized the antinociceptive effects of fentanyl (intraperitoneal), blocking completely the effects of fentanyl up to a dose of 0.1 mg/kg for up to 21 days. Sensitivity to the antinociceptive effects of fentanyl returned to control 45 days after oral administration.

Together, these two studies demonstrate the effectiveness of methocinnamox in antagonizing the effects of the opioid receptor agonist fentanyl after oral administration in rhesus monkeys and rats.

FIG. 2 depicts the apparent bioavailability of MCAM in rats following subcutaneous and oral administration.

FIG. 3 depicts the apparent bioavailability of MCAM in dogs following subcutaneous and oral administration.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds disclosed herein may be provided in the form of pharmaceutically acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

In certain implementations, the methocinnamox is formulated as the hydrochloride salt. In some implementations the methocinnamox is provided as a salt with an organic acid (for instance an alkyl carboxylic acid). In some implementations, the methocinnamox is formulated as the maleate salt. In some implementations, the methocinnamox is formulated as the malate salt. In some implementations, the methocinnamox is formulated as the lactate salt.

As used herein, “opioid overdose” refers to instances in which a subject receives larger quantities of as opioid than may be safely taken. Symptoms arising from opioid overdose can include central nervous system and respiratory depression, hypoxia, miosis, and apnea, any one of which lead to death if not treated rapidly. As such, disclosed herein are methods of treating an overdose in a subject by orally administering methocinnamox to a patient suffering an overdose. The orally active compositions can be used to prevent overdose, for example in cases of accidental exposure in a medical or criminal setting. The orally active compositions can be used prophylactically, for instance if a subject is at risk of being exposed to unsafe levels of opioids (for example an aerosolized opioid chemical agent).

As used herein, a subject refers to an organism receiving the disclosed combinations. In some implementations the subject is a human, for example an adult human, a pediatric human (less than 18 years of age), or a geriatric human (greater than 64 years of age). In some implementations the subject is a domestic animal, for instance a companion animal like a dog or cat, a livestock animal like a cow or pig, a working animal like a horse or donkey, or an exotic animal like an elephant, lion, or tiger.

The orally active compositions may also be used in the treatment of conditions mediated by endogenous opioid agonists (e.g. endorphins), which conditions may be collectively classified together as “endorphin-mediated hedonia,” including but not limited to addictive behaviors like excessive eating (bulimia), drinking (alcoholism), exercise, sex, gambling, etc.

The invention is also directed to a method for treating drug and/or alcohol addiction (involving drug dependency or drug abuse) during withdrawal therapy by administering the orally active compositions to a patient at a dosage sufficient to reduce or eliminate one or more symptoms associated with withdrawal. Such symptoms include nausea, vomiting, anxiety, abdominal cramps, muscle pain, chills and headache. In addition, the combinations decrease the drug cravings typically experienced by addicts after cessation of the abused substance. The orally active compositions are especially useful in the treatment of opioid use disorder such as heroin, morphine, fentanyl, oxycodone, oxymorphone, and methadone (among others). However, it is also useful in treating patients addicted to cocaine, alcohol, amphetamines and combinations of these drugs.

Disclosed herein are methods of treating an opioid use disorder in a subject, by orally administering to the subject an effective amount of methocinnamox, or a pharmaceutically acceptable salt thereof, optionally in combination with at least one additional opioid receptor modulator.

In certain implementations the orally active compositions can be used for treating or preventing opioid dependence. In certain implementations the orally active compositions can be used for reducing or preventing opioid withdrawal symptoms. In some implementations, the orally active compositions can be used for reducing opioid cravings. In some implementations the orally active compositions can be used for treating or preventing opioid overdose, including in some implementations for treating or preventing opioid-associated respiratory depression. In some implementations, the orally active compositions can be used for reducing and/or preventing opioid poisoning. In certain implementations the orally active compositions can be used to treat or prevent opioid addiction. In certain implementations, the orally active compositions can be used to prevent relapse in a recovering addict; by blocking the receptors targeted by opioids, the subject does not experience euphoria upon resumption of opioid use and is thus better able to maintain abstinence from the opioid.

In some implementations the orally active compositions can be used for reducing or preventing one or more signs or symptoms of opioid toxicity. Exemplary symptoms of opioid toxicity include respiratory depression or ventilatory depression, hypoxia, loss of consciousness, decreased respiratory rate, decreased respiratory depth, apnea, delirium, hypotension, bradycardia, decreased body temperature, urinary retention, pupil miosis, sedation, dizziness, nausea, vomiting, constipation, hyperalgesia, immunologic and hormonal dysfunction, muscle rigidity, myoclonus, and bowel dysfunction.

In some implementations the orally active compositions can be used for reducing or preventing renarconization in a patient being treated for opioid overdose. In some implementations, the orally active compositions can be used for treating or preventing opioid overdose, including opioid-associated respiratory depression, by administering the orally active compositions.

In some implementations, the orally active compositions can be used for preventing opioid dependence in a patient receiving an opioid analgesic, and in some implementations the orally active compositions can be used for treating or preventing an opioid-induced adverse event, including opioid overdose, in a patient receiving an opioid analgesic. In some implementations the opioid-induced adverse effect includes one or more of bowel dysfunction, nausea, vomiting, somnolence, dizziness, respiratory depression, headache, dry mouth, sedation, sweats, asthenia, hypotension, dysphoria, delirium, miosis, pruritis, urticaria, urinary retention, hyperalgesia, allodynia, physical dependence, or tolerance. In certain implementations, the orally active compositions can be used for reducing and/or preventing opioid-induced hypoxia.

The orally active compositions can be used in to treat or prevent overdose of any opioid, and in some implementations orally active compositions can be used to treat fentanyl and/or fentanyl analog overdose. Fentanyl analogs are structurally related to fentanyl, with one or more modifications affecting one of more physical and/or pharmacological properties of the drug. Fentanyl analogs are also created in order to avoid detection and scheduling from regulatory and law enforcement regimes. In certain implementations, the fentanyl analog has the formula:

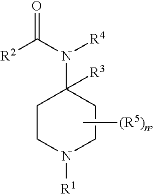

wherein
R¹ is H or C₁₋₆alkyl, optional substituted one or more times by OH, CO₂C₁₋₆alkyl, aryl, C₃₋₁₀cycloalkyl, C₂-10heterocyclyl, or C₁₋₁₀heteroaryl;
R² is C₁₋₆alkyl, C₂-4alkenyl, C₃₋₁₀cycloalkyl or C₁₋₁₀heteroaryl; wherein R² may be substituted by aryl, OC₁₋₆alkyl;
R³ is H, CO₂C₁₋₆alkyl, C₁₋₆alkyl, aryl, wherein R³ may be substituted by OC₁₋₆alkyl;
R⁴ is aryl or C₁₋₁₀heteroaryl, wherein R⁴ substituted one or more times by F;
R⁵ is in each case independently selected from C₁₋₆alkyl, C₂-4alkenyl, and F; and
n is 0, 1, or 2.

In some embodiments, the orally active compositions can be used to treat or prevent overdose of a fentanyl analog such as sufentanil, alfentanil, remifentanil, carfentanil, furanylfentanyl, 4-fluorobutyrylfentanyl, 4-methoxyburtyrylfentanyl, acrylfentanyl, 4-chloroisobutyryfentanyl (4Cl-iBF), 4-fluoroisobutyrfentanyl (4F-iBF), tetrahydrofuranfentanyl (THF-F), cyclopentylfentanyl, AH-7921, U-47700, MT-45, AH-7921, U-47700, AH-7921, or a combination thereof.

The orally active compositions are also useful to treat or prevent overdoses of other opioids, for instance morphine overdose, heroin overdose, oxycodone overdose, hydrocodone overdose, oxymorphone overdose, hydromorphone overdose, codeine overdose, dihydrocodeine overdose, tramadol overdose, buprenorphine overdose, methadone overdose, etorphine overdose or a combination thereof.

The orally active compositions are also used to treat or prevent overdoses with concomitant use of alcohol, barbiturates, xylazine, benzodiazepines, cocaine, amphetamines, gamma-hydroxy butyrate, PCP, ketamine, or a combination thereof.

The orally active compositions can also be used to treat or prevent an alcohol use disorder in a subject, for example to treat or prevent alcohol dependence, to assist with alcohol detoxification, to reduce alcohol cravings, and to treat or prevent alcohol overdose.

The orally active compositions can be used to reduce or prevent alcohol use disorder in an at-risk patient. At-risk patients include those with risk factors that increase the likelihood that alcohol dependence could occur. In some implementations, the at-risk patient is a trauma-survivor. In some implementations the at-risk patient has a family history of alcohol use disorder and/or a personal history of early-onset alcohol use. In some implementations the at-risk patient has received bariatric surgery. In some implementations the at-risk patient has a history of continuous alcohol use that does not rise to the clinical definition of alcohol use disorder.

The orally active compositions can also be used in the long-term treatment of alcohol use disorder. For example the orally active compositions can be used, alone or in combination with supportive counseling, to reduce the level of alcohol consumption in a subject. The orally active compositions can be used, alone or in combination with supportive counseling, in an alcohol-cessation program. The orally active compositions can be administered in conjunctive with other therapeutic agents for alcohol use disorder, including alcohol dehydrogenase inhibitors like disulfiram, NMDA antagonists like acamprosate, or anticonvulsants like gabapentin, pregabalin, or topiramate. The orally active compositions may be used in combinations with antidepressants and other serotonergic agents like odansetron, tropisetron, granisetron, dolasetron, palonosetron, or ramosetron.

In certain implementations, the orally active compositions include methocinnamox, or salt thereof, with one or more opioid receptor modulators, for example opioid agonists, partial agonists, antagonists and allosteric modulators. In some embodiments, the combinations include one or more of naltrexone, naloxone, nalmefene, diprenorphine, nalorphine, nalorphine dinicotinate, levallorphan, samidorphan, nalodiene, buprenorphine, dezocine, eptazocine, butorphanol, levorphanol, nalbuphine, pentazocine, phenazocine, cyprodime, naltrindole, norbinaltorphimine, salts thereof. In certain implementations the disclosed combinations include methocinnamox (or salt thereof) and naltrexone (e.g., naltrexone HCl).

In some implementations, the orally active compositions can include methocinnamox in an amount of 0.5-500 mg, 0.5-250 mg, 0.5-100 mg, 0.5-50 mg, 0.5-25 mg, 0.5-20 mg, 0.5-15 mg, 0.5-10 mg, 0.5-5 mg, 0.5-2.5 mg, 1-2.5 mg, 1-5 mg, 1-10 mg, 2.5-7.5 mg, 5-15 mg, 10-20 mg, 15-25 mg, 20-30 mg, 25-50 mg, 25-75 mg, 50-100 mg, 100-250 mg, or 250-500 mg, wherein the amount is measured as the free base equivalent. In the context of human subjects, the methocinnamox (or salt thereof) can be present in the orally active compositions at a dose level of 0.1-10 mg/kg, 0.1-8 mg/kg, 0.1-6 mg/kg, 0.1-5 mg/kg, 0.1-4 mg/kg, 0.1-3 mg/kg, 0.1-2 mg/kg, 0.1-1 mg/kg, 0.5-1.5 mg/kg, 1-2 mg/kg, 1.5-2.5 mg/kg, 2-5 mg/kg, 2.5-7.5 mg/kg, or 5-mg/kg (calculated as the free base).

In some implementations, the additional opioid receptor modulator, e.g., naltrexone (e.g., naltrexone HCl), can be included in the orally active compositions in an amount of 0.5-100 mg, 0.5-50 mg, 0.5-25 mg, 0.5-20 mg, 0.5-15 mg, 0.5-10 mg, 0.5-5 mg, 0.5-2.5 mg, 1-2.5 mg, 1-5 mg, 1-10 mg, 2.5-7.5 mg, 5-15 mg, 10-20 mg, 15-25 mg, 20-30 mg, 20-40 mg, 25-50 mg, 25-75 mg, or 50-100 mg, wherein the amount is measured as the free base equivalent. In the context of human subjects, the additional opioid receptor modulator, e.g., naltrexone (e.g., naltrexone HCl) can be included in the orally active compositions at a dose level of 0.01-3 mg/kg, 0.01-2 mg/kg, 0.01-1 mg/kg, 0.05-1 mg/kg, 0.1-1 mg/kg, 0.1-0.5 mg/kg, 0.25-0.75 mg/kg, 0.5-1 mg/kg, 0.01-0.1 mg/kg, 0.05-0.1 mg/kg, 0.025-0.075 mg/kg, 0.01-0.05 mg/kg, or 0.075-0.125 mg/kg, wherein the amount is measured as the free base equivalent.

In certain implementations in which methocinnamox is used in combination with one or more additional opioid receptor modulator, the two or more agents can be administered in two or more different dosage forms. In implementations when methocinnamox is administered as a separate composition the agents can be administered simultaneously in two separate compositions. In some implementations the methocinnamox (or salt thereof) and the additional opioid receptor modulator can be administered within a period of 168 hours, 144 hours, 120 hours, 96 hours, 72 hours, 48 hours, 36 hours, 24, hours, 12 hours, 6 hour, 4 hours, 3 hours, 2 hours, 1 hour, 30 minutes, 15 minutes, 10 minutes, 5 minutes, or 2 minutes of each other. In some implementations, the methocinnamox (or salt thereof) is administered before the opioid receptor modulator, while in other implementations the methocinnamox (or salt thereof) is administered after the opioid receptor modulator.

In some implementations the methocinnamox and additional antagonist can be administered according to different dosing schedules. For example, methocinnamox can be administered once every 3 days, once every 5 days, once every 7 days, once every 10 days, or once every 14 days, while the additional antagonists is administered each day, once or twice a day. In some implementation the additional antagonist is provided as a depot formulation for continual release of the antagonist, and the methocinnamox is administered once every 3 days, once every 5 days, once every 7 days, once every 10 days, or once every 14 days.

In some implementations, the period between oral administrations of separate MCAM dosage forms can be at least 3 days, at least 5 days, at last 7 days, at least 10 days, or at least 14 days.

For implementations when the additional opioid receptor modulator is administered separately from the methocinnamox, the additional opioid receptor modulator may be administered enterally, parenterally, intranasally, vaginally, by inhalation, or a combination thereof.

When the additional opioid receptor modulator is administered separately from methocinnamox and enterally, it may be administered by oral administration, sublingual administration, buccal administration, rectal administration, or a combination thereof. When the additional opioid receptor modulator is administered orally, it is especially preferred that the additional opioid receptor modulator is naltrexone (e.g., naltrexone HCl).

When the additional opioid receptor modulator is administered separately from methocinnamox and parenterally, it may be administered by intramuscular injection, intravenous injection, subcutaneous injection, or a combination thereof.

When the additional opioid receptor modulator is administered separately from methocinnamox and intranasally, it is especially preferred that the additional opioid receptor modulator is naloxone (e.g., naloxone HCl).

When the additional opioid receptor modulator is administered separately from methocinnamox and by inhalation, it is especially preferred that the additional opioid receptor modulator is naloxone (e.g. naloxone HCl).

Also disclosed herein are kits for the treatment of an opioid use disorder (as defined herein), or the treatment of an alcohol use disorder (as defined herein). The kits include one or more orally active compositions including methocinnamox, or a pharmaceutically acceptable salt thereof, and at least one additional opioid receptor modulator. In some implementations, the kits include naltrexone, naloxone, nalmefene, diprenorphine, nalorphine, nalorphine dinicotinate, levallorphan, samidorphan, nalodiene, buprenorphine, dezocine, eptazocine, butorphanol, levorphanol, nalbuphine, pentazocine, phenazocine, cyprodime, naltrindole, norbinaltorphimine, salts thereof, or a combination thereof. Especially preferred kits include those containing methocinnamox with naltrexone (e.g., naltrexone HCl) and/or naloxone (e.g., naloxone HCl).

In some implementations, the kits contain methocinnamox, or a salt thereof, and the additional opioid receptor modulator is separate dosage forms. In some implementations, the kits include methocinnamox, or salt thereof, and additional opioid receptor modulator in the same unit dosage form. The kits may include a single dose of methocinnamox, or salt thereof, while in some implementations the kits include multiple doses of methocinnamox, or salt thereof.

The orally active methocinnamox composition may be provide in a variety of dosage forms including tablets, disintegrating tablets, dispersible tablets, granules, capsules (filled with solid dispersion, solid solution, powders, pellets, beads, mini-tablets, pills, micro-pellets, small tablet units, multiple unit pellet systems (MUPS)), sachets (filled with powders, pellets, beads, mini-tablets, pills, micro-pellets, small tablet units, MUPS, disintegrating tablets, dispersible tablets, granules, and microspheres, multiparticulates), powders for reconstitution, controlled release formulations, lyophilized formulations, modified release formulations, delayed release formulations, extended release formulations, pulsatile release formulations, dual release formulations and the like. Liquid or semisolid dosage form (liquids, suspensions, solutions, may also be envisaged under the ambit of the invention.

In certain implementation the methocinnamox (or salt thereof) can be provided as an oral solution or suspension to facilitated administration to very young subjects, very old subjects, and other individuals that have difficulty swallowing solid dosage forms like pills, tablets, capsules etc. The oral solutions and suspensions may further include one or more flavorants or sweetener, or cyclodextrin. In some implementations the methocinnamox is provided as a solid orally administered dosage form for example a tablet or capsule.

In certain implementations, the methocinnamox (or salt thereof) can have an average particle size of less than 200 μm, less than 150 μm, less than 100 μm, less than 50 μm, less than 25 μm, less than 10 μm, less than 5 μm, or less than 2 μm. In certain implementations, the methocinnamox is in the form a maleate salt.

Suitable excipients may be used for formulating the dosage forms according to the present invention such as, but not limited to, surface stabilizers or surfactants, viscosity modifying agents, polymers including extended release polymers, stabilizers, disintegrants or super disintegrants, diluents, plasticizers, binders, glidants, lubricants, sweeteners, flavoring agents, anti-caking agents, opacifiers, anti-microbial agents, antifoaming agents, emulsifiers, buffering agents, coloring agents, carriers, fillers, anti-adherents, solvents, taste-masking agents, preservatives, antioxidants, texture enhancers, channeling agents, coating agents or combinations thereof.

In certain implementations the orally active compositions can include a disintegrant or super disintegrant, for example agar-agar, calcium carbonate, microcrystalline cellulose, crospovidone, povidone, polacrilin potassium, sodium starch glycolate, potato or tapioca starch, other starches, pre-gelatinized starch, clays, alginic acid, alginates such as sodium alginate other algins, other celluloses, gums, ion-exchange resins, magnesium aluminium silicate, sodium dodecyl sulfate, sodium carboxymethyl cellulose, croscarmellose sodium, polyvinyl pyrollidone, cross-linked PVP, carboxymethyl cellulose calcium, cross-linked sodium carboxymethyl cellulose, docusate sodium, guar gum, low-substituted HPC, polacrilin potassium, poloxamer, povidone, sodium glycine carbonate and sodium lauryl sulfate or mixtures thereof. The disintegrant can be present in an amount from about 1% w/w to about 30% w/w, from about 3% w/w to about 15% w/w, or from about 1% w/w to about 10% w/w, of the total weight of the composition. In other implementations, the orally active composition does not include disintegrant.

In certain implementations the orally active compositions can include a binder, for example polyvinyl pyrrolidone (also known as povidone), polyethylene glycol(s), acacia, alginic acid, agar, calcium carrageenan, cellulose derivatives such as ethyl cellulose, methyl cellulose, hydroxypropyl cellulose, hydroxypropyl methyl cellulose, sodium carboxymethyl cellulose, dextrin, gelatin, gum arabic, guar gum, tragacanth, sodium alginate, or mixtures thereof or any other suitable binder. The binder can be present in an amount from about 1% w/w to about 30% w/w, from about 5% w/w to about 20% w/w, of the total weight of the composition. from about 1% w/w to about 5% w/w, from about 5% w/w to about 10% w/w, from about 10% w/w to about 20% w/w, or from about 15% w/w to about 30% w/w.

In certain implementations the orally active compositions can include a carrier, diluent or filler such as lactose (for example, spray-dried lactose, a-lactose, β-lactose) lactose, lactose monohydrate, available under the trade mark Tablettose, various grades of lactose available under the trade mark Pharmatose or other commercially available forms of lactose, lactitol, saccharose, sorbitol, mannitol, dextrates, dextrins, dextrose, maltodextrin, croscarmellose sodium, microcrystalline cellulose (for example, microcrystalline cellulose available under the trade mark Avicel), hydroxypropyl cellulose, L-hydroxypropyl cellulose (low substituted), hydroxypropyl methylcellulose (HPMC), methylcellulose polymers (such as, for example, Methocel A, Methocel A4C, Methocel A15C, Methocel A4M), hydroxyethyl cellulose, sodium carboxymethyl cellulose, carboxymethylene, carboxymethyl hydroxyethyl cellulose and other cellulose derivatives, starches or modified starches (including potato starch, corn starch, maize starch and rice starch) or mixtures thereof. The carriers, diluents and/or filles can be present in an amount from about 10% w/w to about 60% w/w, from about 10% w/w to about 30% w/w, from about 30% w/w to about 60% w/w, from about 45% w/w to about 60% w/w, or from about 20% w/w to about 50% w/w, of the total weight of the composition.

In certain implementations the orally active compositions can include one or more glidants, anti-adherents and lubricants including, but not limited to, stearic acid and pharmaceutically acceptable salts or esters thereof (for example, magnesium stearate, calcium stearate, zinc stearate, sodium stearyl fumarate or other metallic stearate), talc, waxes (for example, microcrystalline waxes) and glycerides, mineral oil, light mineral oil, PEG, silica acid or a derivative or salt thereof (for example, silicates, silicon dioxide, colloidal silicon dioxide and polymers thereof, crospovidone, magnesium aluminosilicate and/or magnesium aluminometasilicate), silicified microcrystalline cellulose such as Prosolv SMCC 90 (silicified microcrystalline cellulose composed of 98% microcrystalline cellulose and 2% colloidal silicon dioxide), sucrose ester of fatty acids, hydrogenated vegetable oils (for example, hydrogenated castor oil, peanut oil, cottonseed oil, sunflower oil, sesame oil, olive oil, corn oil and soybean oil), glycerin, sorbitol, mannitol, other glycols, sodium lauryl sulfate, talc, long chain fatty acids and their salts, ethyl oleate, ethyl laurate, agar, syloid silica gel (a coagulated aerosol of synthetic silica (Evonik Degussa Co., Plano, Tex. USA), a pyrogenic silicon dioxide (CAB-O-SIL, Cabot Co., Boston, Mass. USA), or mixtures thereof. The glidants, anti-adherents and/or lubricants can be present in an amount from about 0.1% w/w to about 5% w/w. In other implementations, the orally active composition does not include a glidant, anti-adherent, or lubricant.

In some implementations, the orally active compositions can include anti-caking additives for example, calcium silicate, magnesium silicate, silicon dioxide, colloidal silicon dioxide, talc, or mixtures thereof. In other implementations, the orally active composition does not include an anti-caking additive.

In some implementations, the orally active compositions can include an antioxidant, for example tocopherols, ascorbic acid, sodium pyrosulfite, butylhydroxytoluene, butylated hydroxyanisole, edetic acid, and edetate salts, or mixtures thereof. In other implementations, the orally active composition does not include an antioxidant.

In some implementations, the orally active compositions can include a texture enhancer for example pectin, polyethylene oxide, and carrageenan, or mixtures thereof. In other implementations, the orally active composition does not include a texture enhancer.

In some implementations, the orally active compositions can include one or more surfactants, including amphoteric surfactants, non-ionic surfactants, cationic surfactants, anionic surfactants, or combination thereof.

In certain implementations, the orally active compositions can include one or more polysorbates, sodium dodecyl sulfate (sodium lauryl sulfate), lauryl dimethyl amine oxide, docusate sodium, cetyltrimethyl ammonium bromide (CTAB) polyethoxylated alcohols, polyoxyethylenesorbitan, octoxynol, N, N-dimethyldodecylamine-N-oxide, hexadecyltrimethylammonium bromide, polyoxyl 10 lauryl ether, Brij, bile salts (sodium deoxycholate, sodium cholate), polyoxyl castor oil, nonylphenolethoxylatecyclodextrins, lecithin, methylbenzethonium chloride. carboxylates, sulphonates, petroleum sulphonates, alkylbenzenesulphonates, naphthalenesulphonates, olefin sulphonates, alkyl sulphates, sulphates, sulphated natural oils, fats, sulphated esters, sulphatedalkanolamides, alkylphenols, ethoxylated and/or sulphated, ethoxylated aliphatic alcohol, polyoxyethylene surfactants, carboxylic esters polyethylene glycol esters, hydrosorbitol ester and ethoxylated derivatives, glycol esters of fatty acids, carboxylic amides, monoalkanolamine condensates, polyoxyethylene fatty acid amides, quaternary ammonium salts, amines with amide linkages, polyoxyethylene alkyl & alicyclic amines, N,N,N,N tetrakis substituted ethylenediamines 2-alkyl 1-hydroxyethyl 2-imidazolines, N-coco 3-aminopropionic acid/sodium salt, N-tallow 3-iminodipropionate disodium salt, N-carboxymethyl n dimethyl n-9 octadecenyl ammonium hydroxide, n-cocoamidethyl n-hydroxyethylglycine sodium salt, Phosal 53 MCT, Tweens, Polyoxyethylene (20) sorbitantrioleate (Tween 85), Oleoylmacrogolglycerides (Labrafil M1944CS), Linoleoylmacrogolglycerides (Labrafil M2125CS), PG monolaurate (Lauroglycol 90), D-alpha-tocopheryl PEG 1000 succinate (Vitamin E TPGS), Polyoxyl 35 castor oil (Cremophor EL, Cremophor ELP), Polyoxyl 40 hydrogenated castor oil (Cremophor RH 40, Cremophor RH 60), Lauroylmacrogolglycerides (Gelucire 44/14, Gelucire 50/13), Lauroyl macrogol-32 glycerides, Lauroyl polyoxyl-32 glycerides, Lauroylpolyoxylglycerides, Caprylocaproylmacrogol glycerides (Labrasol), Polyoxyethylene (20) sorbitanmonooleate, (Polysorbate 80/Tween 80), Polyoxyethylene (20) sorbitanmonolaurate (Polysorbate 20/Tween 20), polyglycerol (polyglyceryloleate: Plural™ Oleique CC497) propylene glycol (propylene glycol monocaprylate: Capryol™ 90, propylene glycol monolaurate: Lauroglycol 90), polyoxyethylene glycols (PEG-8 stearate: Mirj 45, PEG-40 stearate: Mirj® 52, PEG-15 hydroxystearate: Solutol® HS15), sorbitan or monoanhydrosorbitol (sorbitanmonooleate: Span® 80, sucrose (sucrose monopalmitate: Surfhope® D-1616), Lutrol E 300, Transcutol HP, Transcutol P, Soyabean oil, Labrafac PG, Milyol 840, Pluronic L44, Pluronic L64, Polaxamer 188, and the like or mixtures thereof. The orally active compositions may include one or more surfactants in an amount from about 1% w/w to about 15% w/w, from about 2% w/w to about 10% w/w, from about 2% w/w to about 5% w/w, or from about 5% w/w to about 10% w/w, of the total weight of the composition. In other implementations, the orally active composition does not include a surfactant.

In certain implementations, the orally active compositions can include one or more viscosity modifying agents, for example lactose, sucrose, saccharose, hydrolyzed starch (maltodextrin) or mixtures thereof. In some implementations the orally active compositions can include one or more viscosity modifying agents in an amount about 1% w/w to about 25% w/w, about 1% w/w to about 5% w/w, from about 5% w/w to about 20% w/w, about 5% w/w to about 10% w/w, or about 10% w/w to about 20% w/w, of the total weight of the composition. In other implementations, the orally active composition does not include a viscosity modifying agent.

In certain implementations, the orally active compositions can include one or more polymers or polymers blends, for example hydrophilic polymers, such as hydroxypropyl cellulose, hydroxymethyl cellulose, hydroxypropyl methylcellulose, hydroxypropylcellulose acetate-succinate, hydroxypropyl methyl cellulose phthalate, or cellulose acetate phthalate, methylcellulose polymers hydroxyethyl cellulose, sodium carboxymethyl cellulose, carboxymethylene and carboxymethyl hydroxyethyl cellulose; acrylics like acrylic acid, acrylamide, and maleic anhydride polymers, polyvinyl caprolactam-polyvinyl acetate-polyethylene glycol graft copolymer (Soluplus®), polyvinylpyrrolidone/vinyl acetate co-polymer, acacia, gum tragacanth, locust bean gum, guar gum, or karaya gum, agar, pectin, carrageenan, gelatin, casein, zein and alginates, carboxypolymethylene, bentonite, magnesium aluminum silicate, polysaccharides, modified starch derivatives and copolymers or mixtures thereof. In one embodiment, the polymer is hydroxypropyl methyl cellulose acetate. In yet another embodiment, the polymer is hydroxypropyl methyl cellulose acetate succinate (HPMCAS). In yet another embodiment the polymer is polyvinyl caprolactam-polyvinyl acetate-polyethylene glycol graft copolymer (Soluplus®). In yet another embodiment the polymer is polyvinylpyrrolidone/vinyl acetate co-polymer, optionally in combination with polyvinyl caprolactam-polyvinyl acetate-polyethylene glycol graft copolymer (Soluplus®). The orally active compositions may include one or more polymers in an amount from about 1% w/w to about 75% w/w, from about 1% w/w to about 25% w/w, from about 20% w/w to about 40% w/w, from about 25% w/w to about 50% w/w, from about 25% w/w to about 75% w/w, or from about 50% w/w to about 75% w/w, of the total weight of the composition. In other implementations, the orally active composition does not include a polymer.

### EXAMPLES

The following examples are for the purpose of illustration of the invention only and are not intended to limit the scope of the present invention in any manner whatsoever.

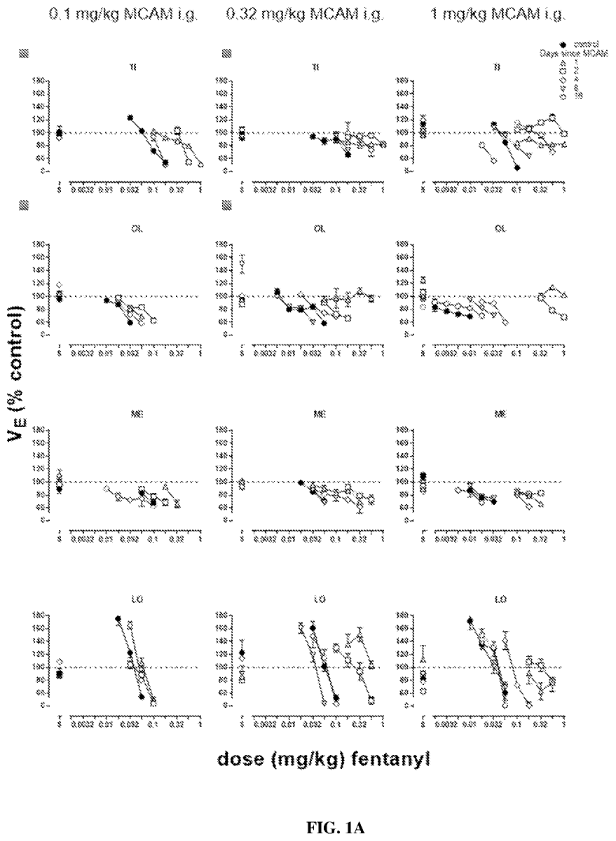

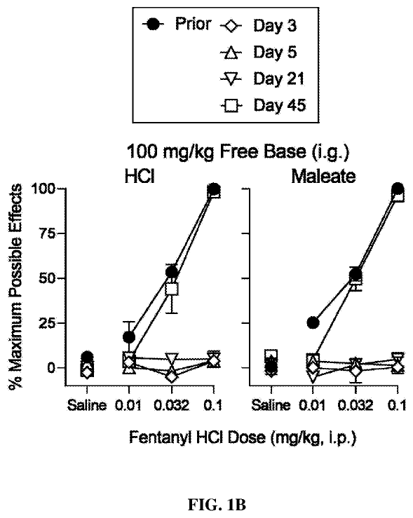

Example 1: Rhesus monkeys sat in chairs equipped with head plethysmography helmets connected and controlled by a data acquisition module (Data Sciences International) and computer. Normal air was pumped in and out of the helmet at a rate of 10 l/minute. Ventilation was monitored and measured by a pneumotachometer that detected pressure changes in the helmet. The primary dependent variables were frequency and volume of ventilation. The product of these two values (frequency x tidal volume) reflects the total inspired volume per minute. Data in FIG. 1A show the total volume of inspiration per minute (i.e., minute volume [V_{E}]) for 4 individual monkeys. In experimental sessions, monkeys received increasing doses of fentanyl (filled circles) up to doses that significantly decreased V_{E}. On other occasions, monkeys received a dose of MCAM (0.1, 0.32, or 1.0 mg/kg) administered by nasogastric intubation with sensitivity to the ventilatory depressant effects of fentanyl assessed 1, 2, 4, 8, and 10 days later in monkeys TI, OL, ME, and LO. Under control conditions, fentanyl decreased ventilation in a dose-related manner (filled circles, all panels, FIG. 1A). MCAM attenuated the ventilatory depressant effects of fentanyl as reflected by shifts to the right in the fentanyl dose-response function and the need for larger doses of fentanyl to significantly decrease V_{E} (open symbols, all panels, FIG. 1A). Shifts in the fentanyl dose-response function were related to MCAM dose, with larger shifts rightward obtained with larger doses of MCAM (1.0 mg/kg; right panels, FIG. 1A). This study demonstrates the effectiveness of orally administered MCAM in blocking the ventilatory depressant effects of fentanyl in rhesus monkeys.

The antinociceptive effects of fentanyl (administered intraperitoneally) were studied in rats under control conditions and after oral administration (intragastric intubation) of 100 mg/kg (free base) of MCAM hydrochloride (HCl) or MCAM maleate. Under control conditions, the time for rats to remove their tails from warm (50° C.) water was increased in a dose-related manner (filled circles, both panels, FIG. 1B; values are the mean and standard effort of the mean for determinations in two rats). MCAM attenuated the antinociceptive effects of fentanyl as reflected by the absence of any increase in tail withdrawal latency up to a dose of 0.1 mg/kg fentanyl. A single dose of orally administered MCAM continued to attenuate the antinociceptive effects of fentanyl for at least 21 days (inverted triangles, FIG. 1B). Sensitivity to the antinociceptive effects of fentanyl returned to control values 45 days after administration of MCAM (squares, FIG. 1B). This study demonstrates the effectiveness of orally administered MCAM in blocking the antinociceptive effects of fentanyl in rats.

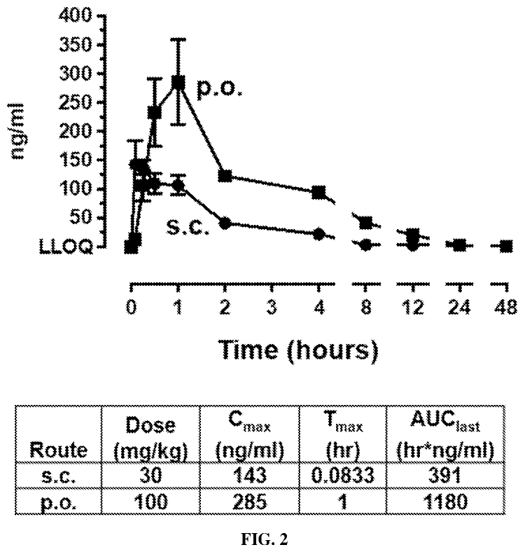

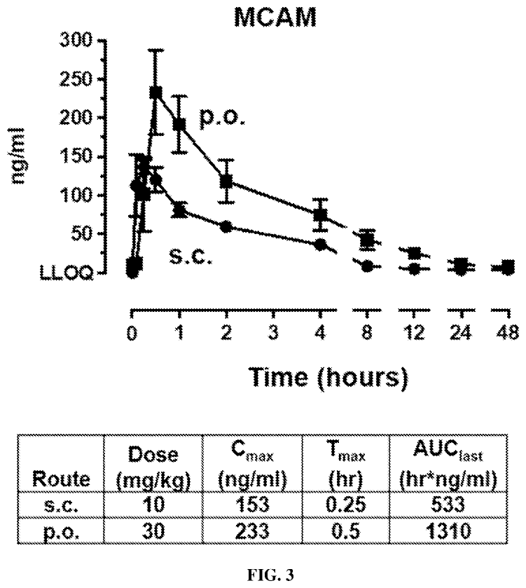

Example 2: The apparent bioavailability of MCAM was determined in rats and dogs. The rat study used separate groups of 6 male rats for s.c. (30 mg/kg) and p.o. (100 mg/kg) dosing. The dog study used the same 4 dogs for s.c. (10 mg/kg) and p.o. (30 mg/kg) dosing. FIGS. 2 and 3 depict plasma concentrations of MCAM (ng/ml) plotted as a function of time (hours) after administration of MCAM. In both studies, a 3-fold increase in dose (from s.c. to p.o.) yielded a nearly 3-fold increase in exposure (area under the curve; AUC).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. An orally administrable composition comprising methocinnamox, or a pharmaceutically acceptable salt thereof, and at least one pharmaceutically acceptable excipient.

2. The composition according to any preceding claim, wherein the methocinnamox is methocinnamox HCl, methocinnamox malate, methocinnamox maleate, or
methocinnamox lactate.

3. The composition according to any preceding claim, wherein the methocinnamox is methocinnamox maleate.

4. The composition according to any preceding claim, wherein the composition comprising methocinnamox, or a pharmaceutically acceptable salt thereof in an amount of 0.5-500 mg, 0.5-250 mg, 0.5-100 mg, 0.5-50 mg, 0.5-25 mg, 0.5-20 mg, 0.5-15 mg, 0.5-10 mg, 0.5-5 mg, 0.5-2.5 mg, 1-2.5 mg, 1-5 mg, 1-10 mg, 2.5-7.5 mg, 5-15 mg, 10-20 mg, 15-25 mg, 20-30 mg, 25-50 mg, 25-75 mg, 50-100 mg, 100-250 mg, or 250-500 mg, wherein the amount is measured as the free base equivalent.

5. The composition according to any preceding claim, comprising methocinnamox, or a pharmaceutically acceptable salt, as an aqueous solution or dispersion.

6. The composition according to any preceding claim, wherein at least one pharmaceutically acceptable excipient has a melting point above 40° C.

7. The composition according to any preceding claim, wherein the methocinnamox, or pharmaceutically acceptable salt thereof, has an average particle size of less than 200 μm, less than 150 μm, less than 100 μm, less than 50 μm, less than 25 μm, less than 10 μm, less than 5 μm, or less than 2 μm.

8. The composition according to any preceding claim, wherein the composition further comprises at least one additional opioid receptor modulator.

9. The composition according to any preceding claim, wherein the additional opioid receptor modulator is naltrexone, naloxone, nalmefene, diprenorphine, nalorphine, nalorphine dinicotinate, levallorphan, samidorphan, nalodiene, buprenorphine, dezocine, eptazocine, butorphanol, levorphanol, nalbuphine, pentazocine, phenazocine, cyprodime, naltrindole, norbinaltorphimine, salts thereof, or a combination thereof.

10. The composition according to any preceding claim, wherein the additional opioid receptor modulator is naltrexone HCl.

11. The composition according to any preceding claim, wherein the composition comprises at least one additional opioid receptor modulator in an amount of 0.5-100 mg, 0.5-50 mg, 0.5-25 mg, 0.5-20 mg, 0.5-15 mg, 0.5-10 mg, 0.5-5 mg, 0.5-2.5 mg, 1-2.5 mg, 1-5 mg, 1-10 mg, 2.5-7.5 mg, 5-15 mg, 10-20 mg, 15-25 mg, 20-30 mg, 25-50 mg, 25-75 mg, or 50-100 mg, wherein the amount is measured as the free base equivalent.

12. The composition according to any preceding claim, wherein the composition comprises naltrexone HCl in an among from 25-75 mg, wherein the amount is measured as the free base equivalent.

13. A method of treating an opioid use disorder, comprising orally administering the composition according to any preceding claim to a subject in need thereof.

14. The method according to any preceding claim, wherein the method is for treating or preventing opioid dependence.

15. The method according to any preceding claim, wherein the method is for reducing or preventing opioid withdrawal symptoms.

16. The method according to any preceding claim, wherein the method is for reducing opioid cravings.

17. The method according to any preceding claim, wherein the method is for treating or preventing opioid overdose.

18. The method according to any preceding claim, wherein the method is for treating or preventing opioid-associated respiratory depression.

19. The method according to any preceding claim, wherein the method is for reducing or preventing renarconization in a patient being treated for opioid overdose.

20. The method according to any preceding claim, wherein the method is for preventing opioid dependence in a patient receiving an opioid analgesic.

21. The method according to any preceding claim, wherein the method is for preventing opioid overdose in a patient receiving a medically prescribed opioid.

22. The method according to any preceding claim, wherein the method is for preventing an opioid-induced adverse event in a patient receiving a medically prescribed opioid.

23. The method according to any preceding claim, wherein the method is for preventing an opioid-induced adverse event in a patient receiving a medically prescribed opioid, wherein the opioid-induced adverse effect comprises bowel dysfunction, nausea, vomiting, somnolence, dizziness, respiratory depression, headache, dry mouth, sedation, sweats, asthenia, hypotension, dysphoria, delirium, miosis, pruritis, urticaria, urinary retention, hyperalgesia, allodynia, physical dependence, tolerance, or a combination thereof.

24. The method according to any preceding claim, comprising reducing and/or preventing opioid-induced hypoxia.

25. The method according to any preceding claim, comprising reducing and/or preventing opioid poisoning.

26. The method according to any preceding claim, wherein the opioid use disorder is fentanyl overdose, fentanyl analog overdose, morphine overdose, heroin overdose, oxycodone overdose, hydrocodone overdose, oxymorphone overdose, hydromorphone overdose, codeine overdose, dihydrocodeine overdose, tramadol overdose, buprenorphine overdose, methadone overdose, etorphine or a combination thereof.

27. The method according to any preceding claim, wherein the fentanyl analog has the structure: 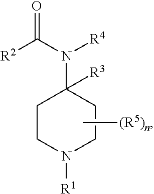
wherein
R¹ is H or C₁₋₆alkyl, optional substituted one or more times by OH, CO₂C₁₋₆alkyl, aryl, C₃₋₁₀cycloalkyl, C₂₋₁₀heterocyclyl, or C₁₋₁₀heteroaryl;
R² is C₁₋₆alkyl, C₂₋₄alkenyl, C₃₋₁₀cycloalkyl or C₁₋₁₀heteroaryl; wherein R² may be substituted by aryl, OC₁₋₆alkyl;
R³ is H, CO₂C₁₋₆alkyl, C₁₋₆alkyl, aryl, wherein R³ may be substituted by OC₁₋₆alkyl;
R⁴ is aryl or C₁₋₁₀heteroaryl, wherein R⁴ substituted one or more times by F;
R⁵ is in each case independently selected from C₁₋₆alkyl, C₂₋₄alkenyl, and F; and
n is 0, 1, or 2.

28. The method according to any preceding claim, wherein the fentanyl analog is sufentanil, alfentanil, remifentanil, and carfentanil, furanylfentanyl, 4-fluorobutyrylfentanyl, 4-methoxyburtyrylfentanyl, acrylfentanyl, 4-chloroisobutyryfentanyl (4Cl-iBF), 4-fluoroisobutyrfentanyl (4F-iBF), tetrahydrofuranfentanyl (THF-F), cyclopentylfentanyl, AH-7921, U-47700, MT-45, AH-7921, U-47700, AH-7921, or a combination thereof.

29. The method according to any preceding claim, wherein the opioid use disorder is fentanyl overdose, fentanyl analog overdose, or a combination thereof.

30. The method according to any preceding, wherein the opioid use disorder is opioid overdose with concomitant use of alcohol, barbiturates, xylazine, benzodiazepines, cocaine, amphetamines, gamma-hydroxy butyrate, PCP, ketamine, or a combination thereof.

31. The method according to any preceding claim, wherein the opioid use disorder is opioid addiction.

32. A method for supporting opioid cessation in a subject in need thereof, comprising orally administering to the subject the composition according to any preceding claim.

33. A method of treating an alcohol use disorder in a subject, comprising orally administering to the subject the composition according to any preceding claim.

34. The method according to any preceding claim, wherein the alcohol use disorder is alcohol dependence.

35. The method according to any preceding claim, wherein the alcohol use disorder is alcohol detoxification.

36. The method according to any preceding claim, wherein the method is for reducing alcohol cravings.

37. The method according to any preceding claim, wherein the method is for treating or preventing alcohol overdose.

38. The method according to any preceding claim, wherein the method is for reducing or preventing alcohol use disorder in an at-risk patient.

39. The method according to any preceding claim, wherein the at-risk patient is a trauma-survivor.

40. A method for reducing alcohol consumption in a subject, comprising orally administering to the subject the composition according to any preceding claim, in combination with at least one additional opioid receptor modulator.

41. A method for promoting alcohol use cessation in a subject, comprising orally administering to the subject the composition according to any preceding claim, in combination with at least one additional opioid receptor modulator.

42. The method according to any preceding claim, further comprising administering an alcohol dehydrogenase inhibitor, NMDA antagonist, anticonvulsant, antidepressant, other serotonergic agent, or combination thereof like.

43. The method according to any preceding claim, wherein the alcohol dehydrogenase inhibitor is disulfiram, the NMDA antagonist is acamprosate, the anticonvulsant is gabapentin, pregabalin, or topiramate.

44. The method according to any preceding claim, wherein the additional opioid receptor modulator is naltrexone, naloxone, nalmefene, diprenorphine, nalorphine, nalorphine dinicotinate, levallorphan, samidorphan, nalodiene, buprenorphine, dezocine, eptazocine, butorphanol, levorphanol, nalbuphine, pentazocine, phenazocine, cyprodime, naltrindole, norbinaltorphimine, salts thereof, or a combination thereof.

45. The method according to any preceding claim, wherein the additional opioid receptor modulator is naltrexone, or a pharmaceutically acceptable salt thereof.

46. The method according to any preceding claim, wherein the additional opioid receptor modulator is naltrexone HCl.

47. The method according to any preceding claim, wherein the additional opioid receptor modulator is naloxone, or a pharmaceutically acceptable salt thereof.

48. The method according to any preceding claim, wherein the additional opioid receptor modulator is naloxone HCl.

49. The method according to any preceding claim, wherein the methocinnamox and the additional opioid receptor modulators are administered in the same event dosing event.

50. The method according to any preceding claim, wherein the composition is administered no more than once a day, no more than once every 3 days, no more than once every 5 days, no more than once every 7 days, no more than once every 10 days, or no more than once every 14 days.

51. The method according to any preceding claim, wherein the methocinnamox is administered before the additional opioid receptor modulator.

52. The method according to any preceding claim, wherein the methocinnamox is administered after the additional opioid receptor modulator.

53. The method according to any preceding claim, wherein the additional opioid receptor modulator is administered enterally by oral administration, sublingual administration, buccal administration, rectal administration, or a combination thereof.

54. The method according to any preceding claim, wherein the additional opioid receptor modulator is administered parenterally by intramuscular injection, intravenous injection, subcutaneous injection, or a combination thereof.

55. The method according to any preceding claim, wherein the additional opioid receptor modulator is administered intranasally.

56. The method according to any preceding claim, wherein the additional opioid receptor modulator is administered by inhalation.

57. The method according to any preceding claim, wherein the methocinnamox and additional opioid receptor modulator are administered in the same unit dose.

58. A kit for the treatment of an opioid use disorder, comprising the composition according to any preceding claim.

59. The kit according to any preceding claim, further comprising at least one additional opioid receptor modulator.

60. The kit according to any preceding claim, wherein the additional opioid receptor modulator is naltrexone, naloxone, nalmefene, diprenorphine, nalorphine, nalorphine dinicotinate, levallorphan, samidorphan, nalodiene, buprenorphine, dezocine, eptazocine, butorphanol, levorphanol, nalbuphine, pentazocine, phenazocine, cyprodime, naltrindole, norbinaltorphimine, salts thereof, or a combination thereof.

61. The kit according to any preceding claim, wherein the additional opioid receptor modulator is naltrexone, or a pharmaceutically acceptable salt thereof.

62. The kit according to any preceding claim, wherein the additional opioid receptor modulator is naltrexone HCl.

63. The kit according to any preceding claim, wherein the additional opioid receptor modulator is naloxone, or a pharmaceutically acceptable salt thereof.

64. The kit according to any preceding claim, wherein the additional opioid receptor modulator is naloxone HCl.

65. The kit according to any preceding claim, wherein the additional opioid receptor modulator is provided as a separate unit dosage form.
